(19) United States
(12) Patent Application Publication
(10) Pub. No.: US 2022/0355046 A1
(43) Pub. Date: Nov. 10, 2022

(54) Implantable vein localizer and methods of accessing a vein using same

(71) Applicant: Farajallah Mounir Hanna al-kass, Glendale, CA (US)

(72) Inventor: Farajallah Mounir Hanna al-kass, Glendale, CA (US)

(21) Appl. No.: 17/734,600

(22) Filed: May 2, 2022

Related U.S. Application Data
(60) Provisional application No. 63/183,809, filed on May 4, 2021.

(51) Int. Cl. A61M 5/42

(52) U.S. Cl. CPC A61M 5/427; A61M 2205/587

(57) ABSTRACT

A method for gaining access to a vein includes implanting a vein localizer above or adjacent an anterior portion of a vein. The vein localizer features a fluorescent coating which emits visible light upon illumination by an external light. This makes visualization easier for a more confident vessel puncture at the first attempt. The invention is designed to reduce failed venipunctures, improve patient comfort and avoid unnecessary time and expense associated with multiple vein punctures.

### CROSS-REFERENCE DATA

The present application claims a priority date benefit from a co-pending U.S. Provisional Patent Application No. 63/183,809 filed 4 May 2021 by the same inventor and entitled “Vein localizer device”, the content of which is incorporated by reference in its entirety.

### BACKGROUND

Without limiting the scope of the invention, its background is described in connection with devices and methods facilitating gaining a vein access. More particularly, the invention describes an implantable vein localizer to mark the location of a vein making it easier to find and puncture the vein correctly on the first attempt, following by inserting an intravenous access device therein.

Placing an intravenous access device is sometimes a challenging task for a medical professional. This may be especially problematic in certain groups of patients such as those who are overweight, underweight, have dark skin, children, and in patients who had frequent intravenous access devices placed previously. Intravenous access devices are usually used either for phlebotomy (drawing blood for laboratory analysis) or for infusion of fluid, medications or various blood products.

All intravenous access devices are inserted using a number of procedural steps necessary to gain access to the target subcutaneous vein. In all such cases a piercing needle is used in order to puncture the blood vessel. The needle diameter is usually measured in gauge units and the selection of the proper size depends on the indication for the device (phlebotomy or infusion), patient's diagnosis (needs frequent transfusions), as well as the patient's size and age. In addition, the choice of a target vein and its location plays a role in choosing which needle size is used. The needle size is increased as the location of the intravenous access moves towards the head of the patient along the arm. This is possible because the vein increases in diameter moving from the wrist to the upper arm and above. For this reason, most of the intravenous access devices are usually placed in the upper extremities and to a lesser degree in the neck and shoulders.

One example of a challenging situation is an obese patient. While in a normal patient, the vein 118 is located relatively close to the skin 110—seen as a distance d in FIG. 2; in the setting of the obese patient, the subcutaneous tissue layer under the skin is usually thicker, as seen illustrated in FIG. 3 as the distance D. As a result, the typically superficial vein 118 is located deeper in reference to the skin 110 which makes it difficult to the medical staff to palpate or visually identify the location of the vein in the process of placing an intravenous access device. As the needle puncture is usually done at an angle α to the skin, greater depth D further exacerbates the increase in the subcutaneous distance 199 for the needle to traverse.

Placement of an intravenous access device usually involves a tourniquet which is tightened circumferentially around the arm few centimeters proximal to the intended puncture site. The practical role of the tourniquet is to occlude the veins at the level of the tourniquet by stopping the outflow of the blood from the arm. This keeps the blood pooling in the veins distal to the tourniquet. The pooling of blood slightly increases the pressure in the vein which distends the vein and makes it more apparent visually and with palpation for the medical staff. In certain groups of patients, the vein cannot not be easily visualized prompting a consult from more experienced personnel or placement of the intravenous access device under an ultrasound guidance.

After the first failed attempt of placing the intravenous access device, not only the patient becomes more uncomfortable secondary to pain, but the procedure also becomes more challenging because hematoma and blood extravasation usually developed at the access site and around the vein. These developed hematoma and blood extravasation make it more difficult to the medical staff to feel or see the vein in subsequent attempts as illustrated in FIG. 1. These complications are also more frequent in patients taking anticoagulation and anti-platelets medications (such as Aspirin or Warfarin) as well as patients with congenital bleeding disorders.

Failure to place the intravenous access device might end up delaying the management and treatment of the patient. In certain medical situations such as patient with chest pain, in labor, having a low blood pressure and so on, this delay may have serious medical consequences. In addition, this delay disrupts the workflow in the office or the hospital, in many cases necessitating a call for a more specialized and experienced staff member to achieve insertion of the device after previous attempts have failed.

Repeated attempts of placement of an intravenous device are not only time and resource consuming, but they are also detrimental to the patient. Failed attempts at one location along the vein may prompt shifting to a new location, typically upstream from the first one. In some extreme cases, the medical professional may choose to place the intravenous access devices in central parts of the body such as the shoulder (subclavian vein) or the neck (jugular vein). These central access points are associated with more serious complications if not done correctly, such as a pneumothorax, or an arterial injury.

A comprehensive review of venous puncture complications is presented in Buowari OY. Complications of venipuncture. Advances in Bioscience and Biotechnology, 2013, 4, 126-128, http://dx.doi.org/10.4236/abb.2013.41A018, incorporated herein by reference.

Prior art devices for illumination of veins are known, for example a device made by AccuVein Inc. (Cold Spring Harbor, N.Y.). While these devices work well for superficial veins in the arm, they do not have sufficient light penetration depth to reliably identify superficial deeper locations, such as those in an upper arm (for example basilic and cephalic veins), There is a need to provide a novel method of vein identification that works for all veins, regardless on how deep the blood vessel is located in the body of the patient.

Intravenous access device placement is the most common invasive procedure done in an office or a hospital-based setting. Even a small percentage of patients with challenging intravenous access and those who require more than one attempt to place the intravenous access device constitute a large overall number of patients that end up with failed intravenous access placement. This makes the venous access problem significant and necessitates development of novel devices and methods facilitating reliable intravenous access that is gained on the first puncturing attempt.

### SUMMARY

Accordingly, it is an object of the present invention to overcome these and other drawbacks of the prior art by providing a novel method for accessing a vein using a vein localizer that facilitates gaining access to the vessel on the first attempt.

It is another object of the present invention to provide a novel method of obtaining access to a vein and inserting an intravenous device which reduces complications associated with a conventional technique of accessing a vein.

It is a further object of the present invention to provide a novel method of venipuncture that is more repeatable than the current state of the art so that access to the vessel can be achieved multiple times over the required treatment period.

It is yet a further object of the present invention to provide a novel method of accessing a vein that facilitates cessation of bleeding upon removal of the intravenous device from the vein.

In general terms, the method for accessing a vein may include the following steps:
a. providing a vein localizer;
   The vein localizer in turn may be formed as a flat sheet of biocompatible flexible substrate, such as a generally rectangular sheet of polyester, Teflon, or silicone material. The substrate may have a fluorescent coating on one side thereof, which may be configured to generate visible light upon illumination thereof by an external light. The substrate may also be configured to allow a needle puncture therethrough, followed by an expansion in response to dilation aimed at enlarging its diameter to accept an intravenous device.
b. implanting the vein localizer along and adjacent an anterior portion of the vein;
   The preferred orientation for such implantation is with the fluorescent coating facing up (away from the vein), such that upon healing, the vein localizer coincides with and follows the track of the subcutaneous location of the vein. In some embodiments, more than one vein localizer may be implanted to extend the marked area suitable for gaining vein access
c. illuminating a general location of the vein with an external light at a wavelength selected to cause the fluorescent coating of the vein localizer to emit visible light;
   Covering the vein with the vein localizer makes identifying vein boundaries easier and more repeatable, even at greater tissue depth.
d. accessing the vein by puncturing through the vein localizer at a location made visible by the light emanated therefrom.

Clear recognition of the vein location makes it more likely to puncture the vein along its middle line, which reduces bleeding complications associated with a puncture next to the side wall of the vessel.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is a side view of an internal portion of an elbow showing a potential location of a hematoma resulting from a failed attempt to gain access to a vein therein,

FIG. 2 is a schematic cross-sectional side view of a vein located underneath a skin in subcutaneous tissue,

FIG. 3 is the same but for an obese patient with a greater depth of the vein in the subcutaneous tissue,

FIG. 4 is a schematic cross-sectional side view of the concept of the present invention,

FIG. 5 shows a diagram of illumination of the vein localizer by an external light,

FIG. 6 is a top view of the first embodiment of the present invention in use,

FIG. 7 is a cross-sectional view of the same,

FIG. 8 is a top view of the second embodiment of the present invention,

FIG. 9 is a top view of the third embodiment of the invention, and

FIG. 10 is a cross-section view of the same.

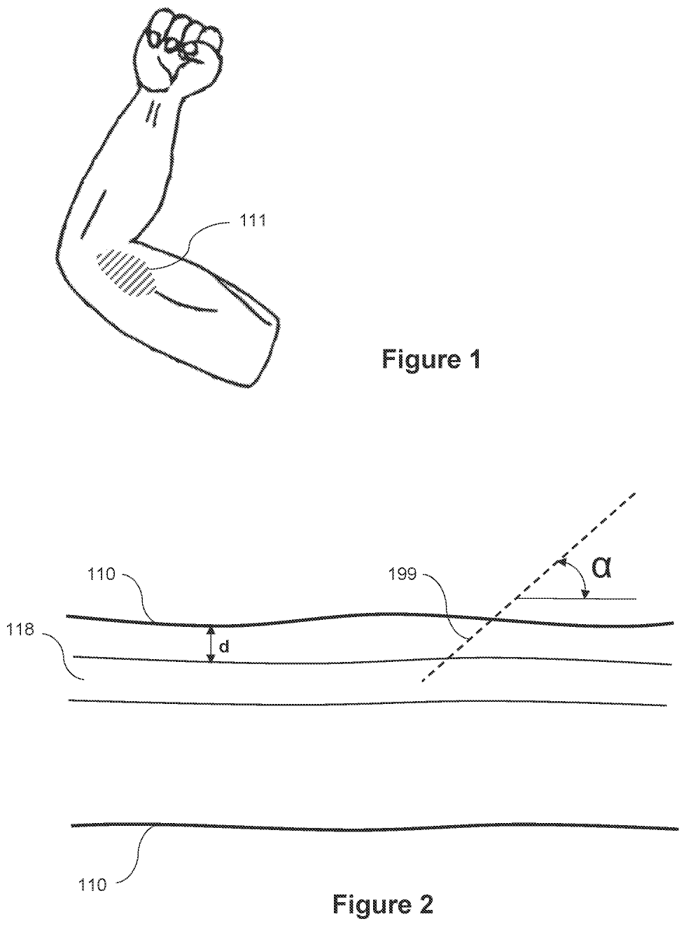

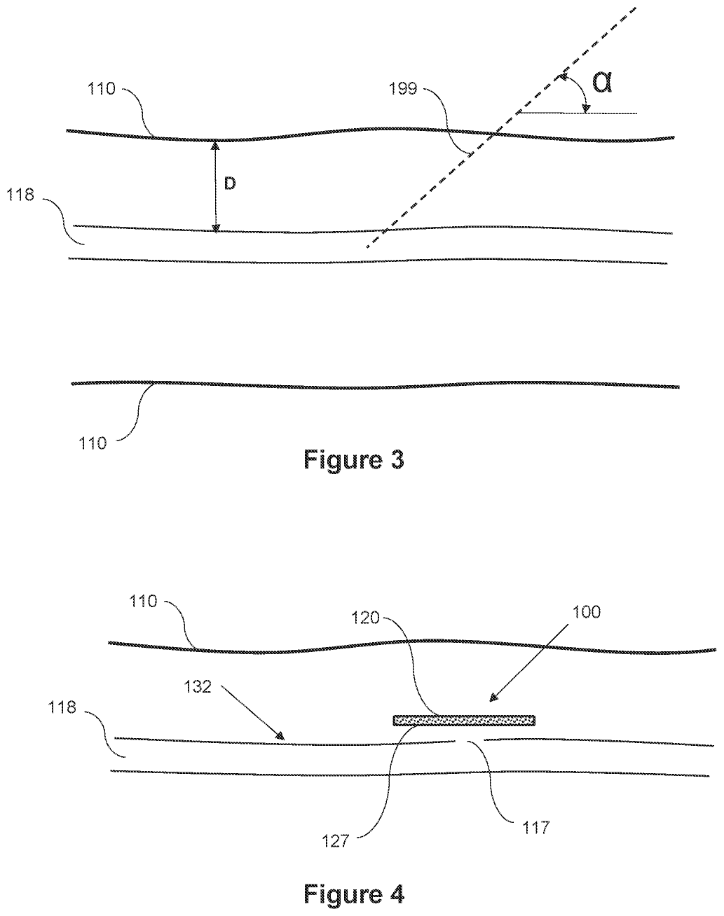

### DETAILED DESCRIPTION OF PREFERRED EMBODIMENTS OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

FIG. 4 shows a side view of the main concept of the invention. The vein localizer 100 is seen implanted above the anterior portion 132 of the vein 118 in the subcutaneous tissue below the skin 110. The vein localizer is made from a flat synthetic sheet of a biocompatible flexible substrate, such as a polyester fabric, a Teflon fabric, a silicone sheet, a polyurethane sheet, a sheet of another biocompatible polymer, or a combination thereof. Material of the substrate may be made to be permanently implantable and stable once inserted in the subcutaneous tissue. In other embodiments, the material of the substrate may be selected to be bioabsorbable after a predetermined period of time, such as at least 1 week, at least 2 weeks, at least 3 weeks, at least 1 month, at least 2 months, at least 3 months, at least 4 months, at least 5 months, or at least 6 months. The advantage of using a stable material is the ability to access the vein multiple times over many years, while the advantage of a bioresorbable material for the substrate is a natural absorption and disappearance of the vein localizer after the intended therapy has ended. Therefore, a suitable substrate may be selected based on clinical indication and specific circumstances for each patient.

The substrate of the vein localizer 100 may have a generally rectangular shape. The width of the vein localizer may be selected to cover the width of the target vein such that multiple sizes of the vein localizer may be provided to match the size of the vessel. The width of the substrate may range from about 3 mm to about 8 mm, such as at least 3 mm, at least 4 mm, at least 5 mm, at least 6 mm, at least 7 mm, or about 8 mm.

The length of the substrate may range from about 10 mm to about 30 mm, such as at least 10 mm, at least 15 mm, at least 20 mm, at least 25 mm, at least 30 mm or even longer as the invention is not limited in this regard.

In further embodiments, the size of the substrate may be even larger than mentioned above such that the vein localizer may be trimmed to size during the implantation procedure.

The substrate may be made to be flexible and thin, such as from about 0.3 mm to about 2 mm thick.

The substrate may further be made from a material that allows an easy puncture with a needle. It is assumed that needle puncture will be performed during every procedure of gaining access to the vein so that the substrate material should not cause appreciable resistance to the needle penetrating across thereof.

A further feature of the substrate material may be its ability to stretch without significant effort, at least to an extent to allow enlargement of a needle opening by a conventional vessel dilator. This feature is useful in allowing positioning of an intravenous device such as a cannula or a catheter that exceeds the size of the initial needle puncture.

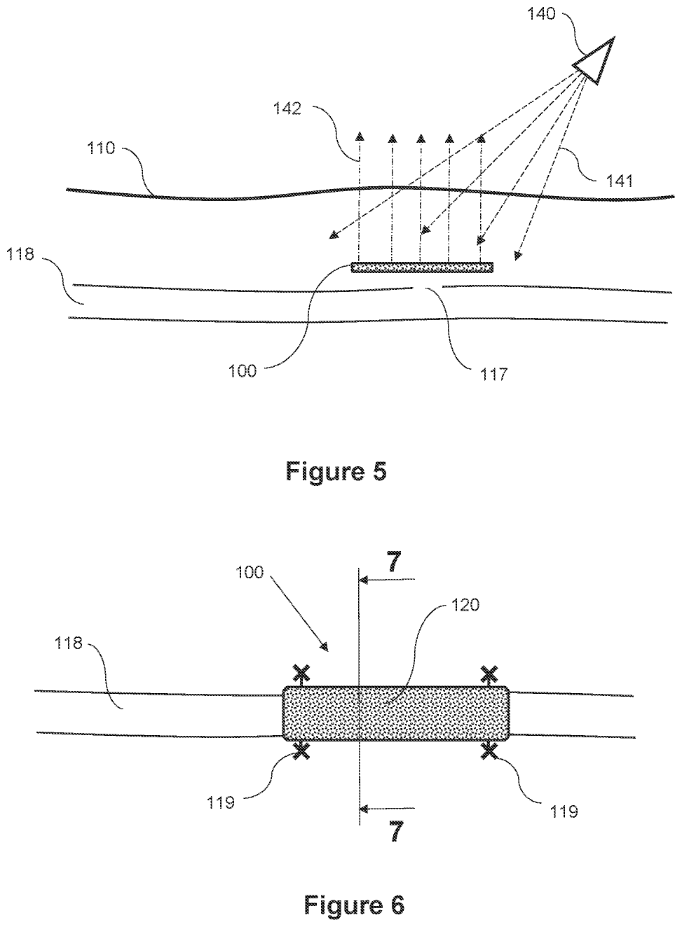

One side of the substrate may be coated with a fluorescent dye 120. A variety of biocompatible, photo-stable fluorescent dies may be used for this purpose. Some examples of a suitable fluorescent coating families of materials include Rhodamines, Coumarins, and Fluoresceins—these materials can emit visible light 142 upon receiving illumination of a suitable wavelength 141 from an external light 140 as seen in FIG. 5. In embodiments, the fluorescent dye of the fluorescent coating may emit visible light at a wavelength of about 500 nm which is sufficiently strong to allow visualization thereof under the skin of the patient. In some embodiments, the external light 140 is a source of ultraviolet irradiation in the range from about 300 nm to about 400 nm.

The opposite side of the substrate of the vein localizer 100 may be coated with a hemostatic material 127, configured to facilitate clotting of liquid blood and closure of the punctured vessel upon removal of the intravenous device therefrom. After removal of the intravenous access device, external pressure is usually applied at the puncture site to stop the bleeding from the venipuncture 117 illustrated in FIG. 4. In this embodiment, applying external pressure on the skin causes the posterior surface of the localizer 127 to get closer to the vessel puncture 117. Once blood mixes with the hemostatic agent of the hemostatic coating 127, clot formation is expedited which helps to stop the bleeding. Using the vein localizer equipped with a hemostatic coating 127 is especially beneficial for patients taking anticoagulant and/or anti-platelet medications, as well as for patients who have congenital bleeding disorders such as Hemophilia A or B and Von Willebrand diseases.

The hemostatic agent used for the hemostatic coating 127 may include a plant- or cellulose-based, a gelatin-based, a collagen-based, a fibrin-based, a thrombin-based, a chitosan-based, a mineral-based material, or a combination thereof. The hemostatic agent may be selected to contain chemical compounds aiding in the blood coagulation process and clot formation, that are critical for obtaining hemostasis and stop the bleeding at the puncture site.

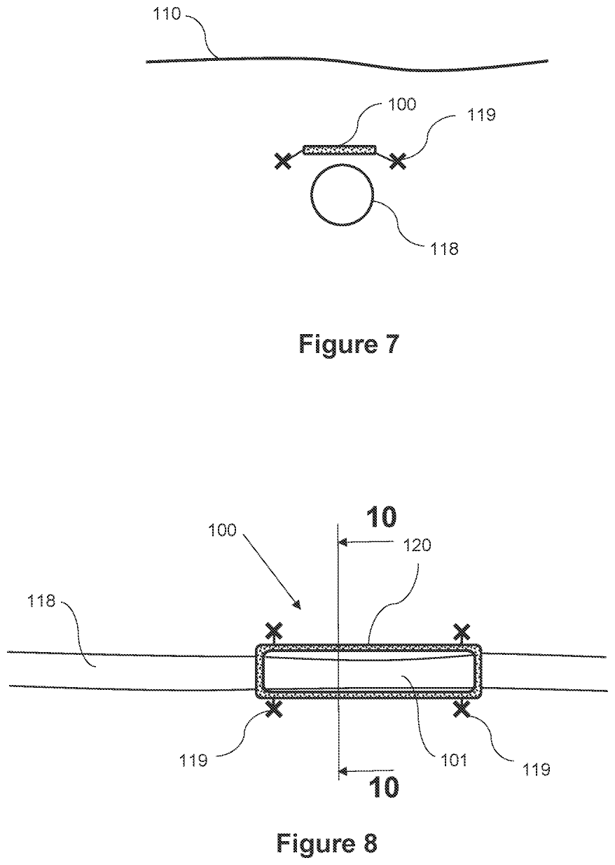

FIGS. 6 and 7 shows a top view and a cross-sectional view of the first embodiment of the invention. Implantation of the vein localizer 100 may be accomplished by using at least some of the following steps:
1. Perform ultrasound surveillance of the vein to verify the patency thereof and general suitability for the intended indication of the intravenous device.
2. Mark the skin for cutdown over the selected vein segment.
3. Under sterile technique and after administration of local anesthesia (such as Lidocaine 2%), make a small skin incision of 1-2 cm, followed by subcutaneous tissue dissection to expose the vein segment.
4. Tighten a tourniquet around the arm proximal to the surgical incision.
5. Measure the size of the vein, select a vein localizer of suitable size or trim a larger vein localizer to size.
6. Position the vein localizer over the vein, such as to cover the boundaries of the vein with the vein localizer. Orient the fluorescent coating up and away from the vein.
7. Secure the vein localizer with sutures 119 to adjacent subcutaneous tissues, such as on all four corners thereof, Staples or glue may also be used as an alternative to the sutures 119.
8. Close the skin incision with absorbable sutures.

More than one vein localizer may be implanted end-to-end so as to increase the marked length of the vein, Implantation of multiple vein localizers may also be useful when the trajectory of the vein is not straight, in which case several straight segments of the individual vein localizers may be used to approximate the curved profile of the underlying blood vessel.

FIG. 8 shows a top view of a second embodiment of the invention in which the vein localizer 100 has a cut-out window 101. Viewing the rectangular outline of the outer boundaries of the vein localizer allow the medical practitioner to puncture the vein 118 within the window 101 such that only the vein 118 is punctured and not the vein localizer 100. A side cross-sectional view is seen in FIG. 10.

The method of gaining access to a vein according to the present invention may be practiced by following the following steps:
a. providing a vein localizer comprising a flat sheet of a flexible biocompatible substrate having a fluorescent coating on one side thereof, the substrate is configured to allow a needle puncture therethrough,
b. implanting the vein localizer along and adjacent an anterior portion of the vein with the fluorescent coating facing away from the vein, wherein upon healing the vein localizer coincides with subcutaneous location of the vein,
c. illuminating a general location of the vein with a light at a wavelength selected to cause the fluorescent coating of the vein localizer to emit visible light, and
d. accessing the vein by puncturing through the vein localizer at a location made visible by the light emanated therefrom.

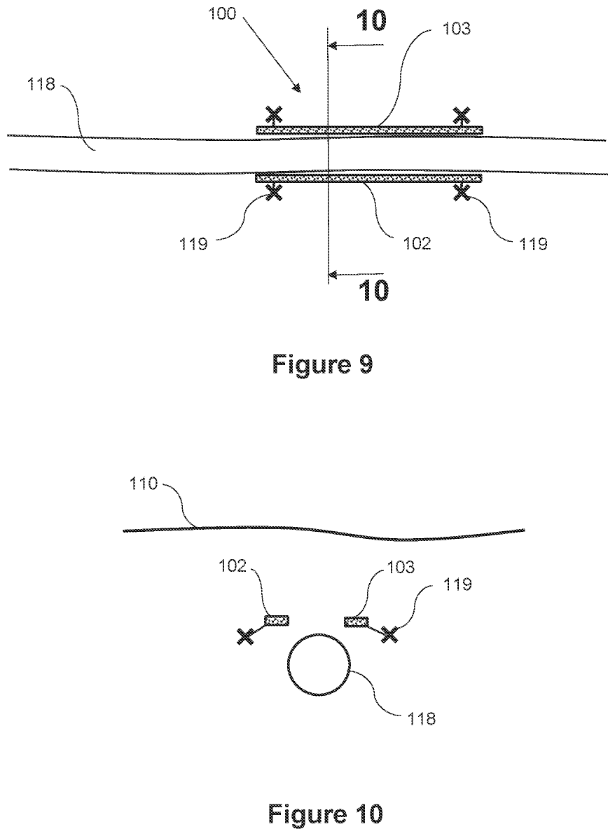

FIG. 9 shows another embodiment of the invention in which the vein localizer 100 consists of a first strip 102 and a second strip 103 positioned next to the outer walls of the vein 118. In cross-section, viewed in FIG. 10, there two strips 102 and 103 located on both sides of the vein 118. The method of gaining an access to the vein in this case may include the following steps:
a. providing a vein localizer comprising a first strip 102 of a flexible biocompatible substrate having a fluorescent coating on one side thereof,
b. implanting the first strip 102 of the vein localizer along and adjacent a first side of an anterior portion of the vein with the fluorescent coating facing away from the vein, wherein upon healing the vein localizer coincides with subcutaneous location of the vein,
c. illuminating a general location of the vein with a light at a wavelength selected to cause the fluorescent coating of the vein localizer to emit visible light, and
d. accessing the vein by puncturing next to the vein localizer at a location made visible by the light emanated therefrom.

When using a second strip, the method further comprises providing a second strip 103 of a flexible biocompatible substrate having a fluorescent coating on one side thereof, the implantation step further comprises a step of implanting the second strip 103 generally parallel with the first strip 102 and adjacent a second side of the anterior portion of the vein.

Observing both strips 102 and 103 allows the medical practitioner to puncture the vein 118 located in between the two strips. Each strip may be independently placed along the wall of the blood vessel during a single implantation procedure.

As compared to the prior art, the novel method of the invention allows reliable identification of various blood vessels, including both superficial veins as well as veins located deeper in the body. This is achieved by the light emanating from the vein localizer, and not just the from external light like in previously known devices.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein, Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Incorporation by reference is limited such that no subject matter is incorporated that is contrary to the explicit disclosure herein, no claims included in the documents are incorporated by reference herein, and any definitions provided in the documents are not incorporated by reference herein unless expressly included herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BOA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims,

What is claimed is:

1. A method for accessing a vein comprising the following steps:
a. providing a vein localizer comprising a flat sheet of a flexible biocompatible substrate having a fluorescent coating on one side thereof, the substrate is configured to allow a needle puncture therethrough,
b. implanting the vein localizer along and adjacent an anterior portion of the vein with the fluorescent coating facing away from the vein, wherein upon healing the vein localizer coincides with subcutaneous location of the vein,
c. illuminating a general location of the vein with a light at a wavelength selected to cause the fluorescent coating of the vein localizer to emit visible light, and
d. accessing the vein by puncturing through the vein localizer at a location made visible by the light emanated therefrom.

2. The method as in claim 1, wherein the vein localizer of step (a) is made in a generally rectangular shape.

3. The method as in claim 2, wherein the vein localizer of step (a) has a width from 3 mm to 8 mm.

4. The method as in claim wherein the vein localizer in step (a) has a length from 10 mm to 30 mm.

5. The method as in claim 1, wherein the substrate of the vein localizer in step (a) is made from an expandable material selected to allow dilation following initial needle puncture to accept an intravenous device placed in the vein therethrough.

6. The method as in claim 1, wherein the substrate of the vein localizer in step (a) is made from a polyester fabric, a Teflon fabric, a sheet of silicone, a sheet of polyurethane, or a combination thereof.

7. The method as in claim 1, wherein the substrate of the vein localizer in step (a) is made from a bioresorbable material.

8. The method as in claim 7, wherein the substrate of the vein localizer in step (a) is made from the bioresorbable material configured to be fully absorbed after at least 1 month post implantation in step (b).

9. The method as in claim 7, wherein the substrate of the vein localizer in step (a) is made from the bioresorbable material configured to be fully absorbed after at least 6 months post implantation in step (b).

10. The method as in claim 1, wherein the substrate of the vein localizer is from 0.3 mm to 2 mm thick.

11. The method as in claim 1, wherein the substrate of the vein localizer has a hemostatic coating on the side opposite to the side with the fluorescent coating, thereby facilitating cessation of bleeding at the puncture site upon removal of the needle or the intravenous device therefrom.

12. The method as in claim 11, wherein the hemostatic coating is cellulose-based, gelatin-based, collagen-based, fibrin-based, thrombin-based, chitosan-based, or mineral-based.

13. The method as in claim 1, wherein step (b) further includes a step of implanting additional vein localizers to follow the vein and increase accessible portion thereof suitable for needle puncturing.

11. The method as in claim 13, wherein each vein localizer is implanted in step (b) to be adjacent to another implanted vein localizer.

12. The method as in claim 1, wherein the vein localizer in step (a) is made with a cut-out window, and wherein in step (b) the implantation of the vein localizer is accomplished by positioning the cut-out window over the vein, thereby facilitating vein puncture through the cut-out window of the vein sterilizer.

13. A method for accessing a v n composing the following steps:
a. providing a vein localizer comprising a first strip of a flexible biocompatible substrate having a fluorescent coating on one side thereof,
b. implanting the first strip of the vein localizer along and adjacent a first side of an anterior portion of the vein with the fluorescent coating facing away from the vein, wherein upon healing the vein localizer coincides with subcutaneous location of the vein,
c. illuminating a general location of the vein with a light at a wavelength selected to cause the fluorescent coating of the vein localizer to emit visible light, and
d. accessing the vein by puncturing next to the vein localizer at a location made visible by the light emanated therefrom.

14. The method as in claim 13, wherein step (a) further comprises providing a second strip of a flexible biocompatible substrate having a fluorescent coating on one side thereof, said step (b) further comprises a step of implanting the second strip generally parallel with the first strip, the second strip is located adjacent a second side of the anterior portion of the vein.
